(19) United States
(12) Patent Application Publication
(10) Pub. No.: US 2018/0360817 A1
(43) Pub. Date: Dec. 20, 2018

(54) EXTENDED RELEASE OSMOTIC DOSAGE FORM COMPRISING HIGH METHYLPHENIDATE HYDROCHLORIDE LOAD

(71) Applicant: CADILA HEALTHCARE LIMITED, AHMEDABAD, GUJARAT (IN)

(72) Inventors: Manish CHAWLA, Ahmedabad (IN); Shailesh Vishwanath BIRADAR, Ahmedabad (IN); Mayur LOYA, Ahmedabad (IN); Jayendrakumar Dasharathlal PATEL, Ahmedabad (IN); Akshay Virendra AGARWAL, Ahmedabad (IN)

(73) Assignee: CADILA HEALTHCARE LIMITED, AHMEDABAD, GUJARAT (IN)

(21) Appl. No.: 16/066,289

(22) PCT Filed: Dec. 30, 2016
(86) PCT No.: PCT/IB2016/058103

§ 371 Date: Jun. 26, 2018

(30) Foreign Application Priority Data
Dec. 31, 2015 (IN) 4977/MUM/2015

(51) Int. Cl. A61K 31/4458; A61K 9/00; A61K 9/20; A61K 9/24

(52) U.S. Cl. CPC A61K 31/4458; A61K 9/0004; A61K 47/10; A61K 9/209; A61K 9/2031

(57) ABSTRACT

The present invention provides an extended release osmotic tablet dosage form comprising methylphenidate hydrochloride (MPH). The dosage form comprises a core tablet comprising methylphenidate hydrochloride in an amount of more than 8% by weight of the core tablet; a semi-permeable membrane surrounding the core tablet, and an immediate release drug coat comprising methylphenidate hydrochloride surrounding the semipermeable membrane.

### FIELD OF THE INVENTION

The present invention relates to an extended release osmotic tablet dosage form comprising methylphenidate hydrochloride (MPH) having high drug load, reduced size and reduced weight in comparison to the commercially available methylphenidate hydrochloride containing osmotic tablet currently marketed in the United States of America (USA) under the trade name Concerta®. The present invention also relates to the process for preparing such methylphenidate hydrochloride containing dosage form.

### BACKGROUND OF THE INVENTION

Methylphenidate hydrochloride is a central nervous system (CNS) stimulant used for Attention Deficit Hyperactivity Disorder (ADHD). The mode of therapeutic action in Attention Deficit Hyperactivity Disorder (ADHD) is not known. Methylphenidate hydrochloride is thought to block the reuptake of norepinephrine and dopamine into the presynaptic neuron and increase the release of these monoamines into the extraneuronal space. Its chemical name is d,l (racemic) methyl α-phenyl-2-piperidineacetate hydrochloride and the structural formula is:

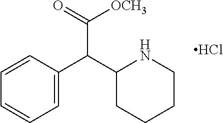 [SMILES: COC(=O)C(c1ccccc1)C1CCCCN1 ; Cl]

Methylphenidate hydrochloride is freely soluble in water and in methanol, soluble in alcohol, and slightly soluble in chloroform and in acetone. Methylphenidate hydrochloride is having molecular weight 269.77 and melting point 204-206° C. Methylphenidate hydrochloride belongs to BCS class I.

U.S. Pat. No. 6,919,373 refers to an osmotic tablet dosage form comprising methylphenidate hydrochloride. It discloses tablets comprising bilayer or trilayer osmotic core tablet having low methylphenidate hydrochloride load.

U.S. Pat. No. 7,674,480 refers to various gastric retention delivery systems and also relates to the timed pulsed release dosage forms. It discloses methylphenidate containing tablets having a matrix core coated with ethyl cellulose and other ingredients to provide the timed pulsed delivery of methylphenidate.

U.S. Pat. No. 7,988,993 discloses a controlled release dosage form having a matrix core tablet comprising methylphenidate coated with acrylic acid based co-polymer to provide once daily tablet dosage form.

US Application No. 2009/0110728 refers to zero-order modified release solid dosage forms comprising methylphenidate. It discloses modified release solid dosage form of methylphenidate having a matrix core coated with ethyl cellulose containing modified release coat.

US Application No. 2012/0189695 discloses methylphenidate containing extended release dosage forms. It discloses extended release tablet dosage forms prepared using the multiparticulates. Each multiparticulate comprise sugar sphere core further coated with the multiple layers like a methylphenidate containing layer, a sustained release layer, a controlled release layer, and then after the multiparticulates are compressed to make the tablet. Compressed tablet is further coated to make a seal coat which is further coated with a methylphenidate containing immediate release coat.

International Publication No. (PCT) WO2006/046114 refers to an osmotic dosage form providing ascending drug release and its preparation. It discloses the osmotic tablet comprising methylphenidate, sorbitol and various other ingredients in a broad range amounts.

Methylphenidate hydrochloride for oral use is commercially available as the osmotic tablet dosage form and currently marketed under the trade name Concerta® by JANSSEN PHARMS.

The present disclosure relates to method for treating Attention Deficit Hyperactivity Disorder (ADHD) in children 6 years of age and older, adolescents, and adults up to the age of 65 using the extended release osmotic tablet dosage forms comprising methylphenidate hydrochloride.

It has been found that the extended release osmotic tablet dosage forms having high methylphenidate hydrochloride load, reduced size and reduced weight can be prepared while maintaining the similar or comparable dissolution profile and bioequivalence parameters. The instant invention provides patient compliance and easy to swallow reduced sized tablet dosage form comprising methylphenidate hydrochloride, especially for pediatric and geriatric ADHD patients who are having difficulties in swallowing the tablets.

It has also been found that the extended release osmotic tablet dosage form of the instant invention provides a tablet having methylphenidate hydrochloride in an amount higher than present in the currently commercially available highest strength of methylphenidate hydrochloride Concerta® tablet (which is 54 mg MPH containing tablet) and still the tablet of invention remains reduced sized and easy to be swallowed.

### SUMMARY OF THE INVENTION

In one general aspect there is provided an extended release osmotic tablet dosage form comprising methylphenidate hydrochloride wherein the tablet is having reduced size and reduced weight in comparison to the commercially available methylphenidate hydrochloride containing extended release osmotic tablet currently marketed under the trade name Concerta®.

In another general aspect there is provided an extended release osmotic tablet dosage form comprising methylphenidate hydrochloride, wherein the osmotic tablet comprises a multilayer core tablet, preferably bilayer or trilayer core tablet, coated with a semipermeable membrane having at least one orifice into it for release of the methylphenidate hydrochloride from the core tablet which is further coated with the drug containing immediate release drug coat.

In another general aspect there is provided an extended release osmotic tablet dosage form comprising methylphenidate hydrochloride, wherein an amount of methylphenidate hydrochloride is more than 8% by weight of the dosage form, for example, more than about 10%, more than about 12% or about 15% by total weight of the dosage form.

In another general aspect there is provided an extended release osmotic tablet dosage form comprising methylphenidate hydrochloride, wherein an amount of methylphenidate hydrochloride is more than 8% by weight of the core tablet, for example, more than about 10%, more than about 12% or about 15% by weight of the core tablet.

In another general aspect there is provided an extended release osmotic tablet dosage form comprising methylphenidate hydrochloride, wherein the tablet comprises methylphenidate hydrochloride as an active ingredient present in the core tablet as well as present in the immediate release drug coat.

In another general aspect there is provided an extended release osmotic tablet dosage form comprising methylphenidate hydrochloride, wherein the core tablet is a multilayer tablet which comprises at least one drug layer and at least one push layer. In one of the embodiments, the push layer does not contain methylphenidate hydrochloride.

Currently, under the tradename Concerta®, 18 mg, 27 mg, 36 mg & 54 mg methylphenidate hydrochloride containing extended release osmotic tablets are approved by USFDA and are commercially available. In one general aspect there is provided an easy to swallow extended release osmotic tablet dosage form comprising methylphenidate hydrochloride in an amount higher than that present in the commercially available tablets under the tradename Concerta®, preferably higher than 54 mg methylphenidate hydrochloride.

In yet another general aspect there is provided a process for preparation of the extended release osmotic tablets comprising methylphenidate hydrochloride, wherein the osmotic tablet is prepared by a process comprising the steps of:

(a) preparing a bilayer core tablet;

(b) applying a semipermeable membrane surrounding the core tablet; and

(c) optionally applying an immediate release drug coat comprising methylphenidate hydrochloride surrounding the semipermeable membrane.

In yet another general aspect there is provided a process for preparation of the extended release osmotic tablets comprising methylphenidate hydrochloride, wherein the osmotic tablet is prepared by a process comprising the steps of:

(a) preparing a drug layer;

(b) preparing a push layer;

(c) longitudinal compression of above two layers of step (a) & step (b) to form a bilayer core tablet;

(d) applying a semipermeable membrane surrounding the core tablet;

(e) creating at least one orifice through the semipermeable membrane;

(f) applying an immediate release drug coat comprising methylphenidate hydrochloride surrounding the semipermeable membrane and

(g) applying an aesthetic film coat surrounding the immediate release drug coat.

Embodiments of the extended release osmotic tablet dosage forms may include one or more of the following features. For example, the extended release osmotic tablet dosage form may further include one or more pharmaceutically acceptable excipients. The pharmaceutically acceptable excipients may include one or more fillers, binders, disintegrants, lubricants, glidants, release rate controlling polymers, osmogents, anti-oxidants, pH modifiers, coating polymers, pore formers, colorants, moisture adsorbents/absorbents, wetting agents, plasticizers and the like.

In yet another general aspect there is provided a method for treatment of Attention Deficit Hyperactivity Disorder (ADHD) comprising administering to the patient in need thereof, the extended release osmotic tablet dosage form comprising methylphenidate hydrochloride as per the invention.

The details of one or more embodiments of the invention are set forth in the description below. Other features of the invention will be apparent from the description.

### BRIEF DESCRIPTION OF THE DRAWINGS

FIG. 1 is schematic diagram for the extended release osmotic tablet dosage form according to the invention to provide tablet dimensions, wherein (a) is thickness of the tablet, (b) is diameter of the tablet and (c) shows narrow ends of the compartments of the tablet.

FIG. 2 is a graph illustrating the in vitro release of methylphenidate hydrochloride over the time following oral administration of the tablet prepared in accordance with Example 1 (round) in comparison to the same for the reference dosage form (triangle).

FIG. 3 is a graph illustrating the plasma methylphenidate hydrochloride concentration over the time following oral administration of the tablet prepared in accordance with Example 1 (round) in fasted condition in comparison to the same for the reference dosage form (triangle) in healthy volunteers.

FIG. 4 schematic diagram for the extended release osmotic tablet dosage form according to the invention to provide cross-sectional view of the tablet; (a) drug layer, (b) push layer, (c) semipermeable membrane, (d) immediate release drug coat and (e) (optional) aesthetic film coat (f) orifice through semipermeable membrane and (g) core tablet

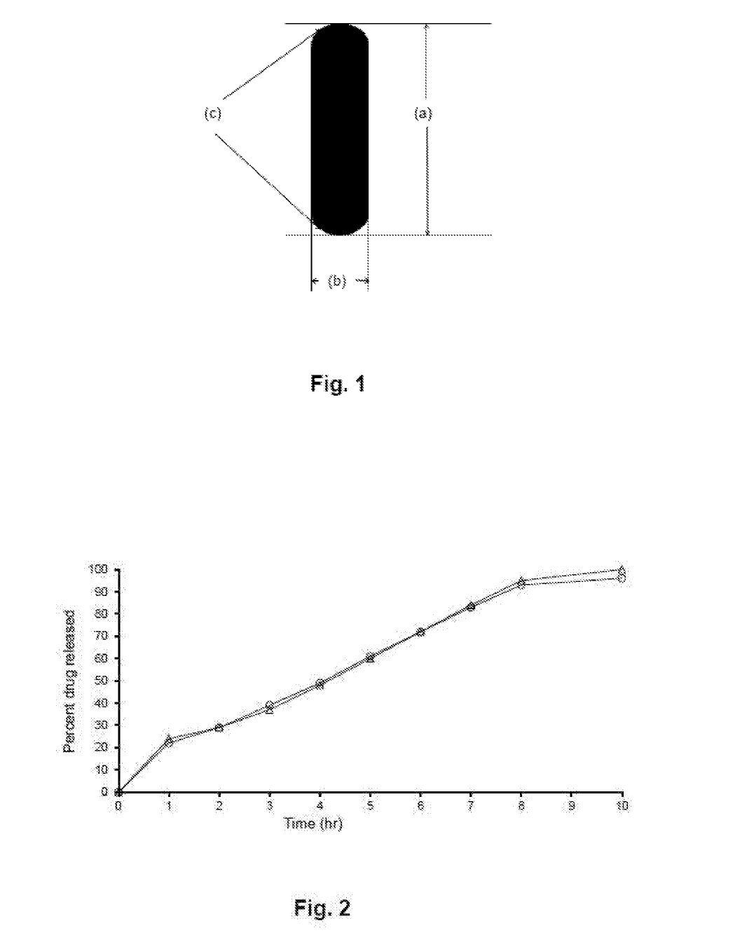

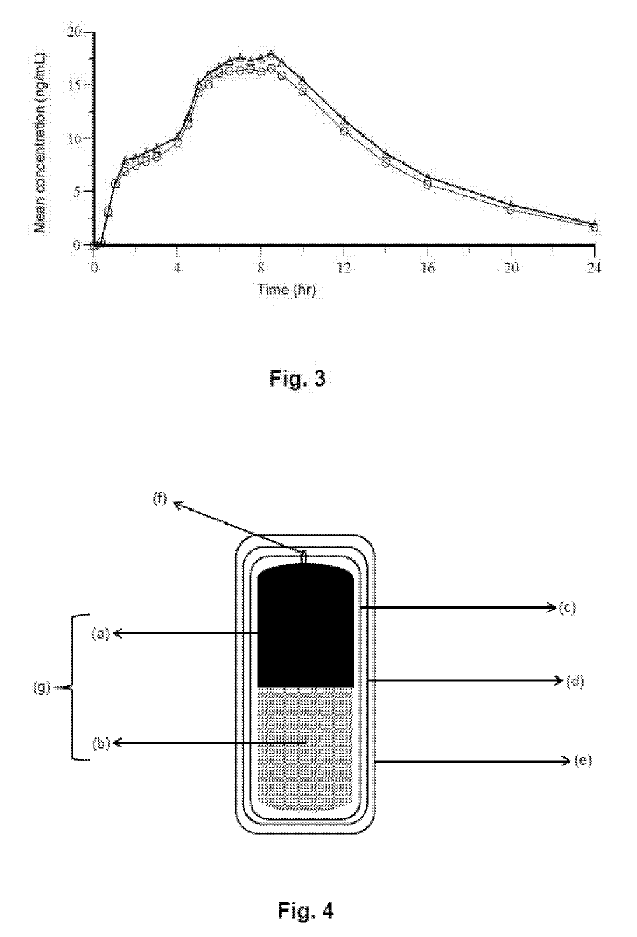

### DETAILED DESCRIPTION OF THE INVENTION

The dosage forms of the present invention are extended release osmotic tablets comprising high drug load, reduced size and reduced weight.

The drug used in present invention is methylphenidate or its pharmaceutically acceptable salts thereof like methylphenidate hydrochloride.

The dosage form of the present invention is bioequivalent to the commercially available methylphenidate hydrochloride containing osmotic tablet currently marketed under the trade name Concerta®.

For the purposes of the present invention, the term “reference dosage form” is intended to mean a tablet dosage form of methylphenidate hydrochloride which is currently approved for marketing and commercially available under the trade name Concerta® in the USA. Unless stated otherwise, for the comparison of parameters like drug load, tablet size, tablet weight, tablet volume, bio-equivalence parameter etc., the dosage form of the present invention comprising specific amount of the methylphenidate hydrochloride is to be compared with the reference dosage form comprising the identical or substantially similar amount of the methylphenidate hydrochloride. For the comparison of the parameters, the dosage form of the present invention comprising 18 mg, 27 mg, 36 mg or 54 mg methylphenidate hydrochloride is to be compared with the reference dosage form comprising 18 mg, 27 mg, 36 mg or 54 mg methylphenidate hydrochloride, respectively.

For the purposes of the present invention, the term “methylphenidate hydrochloride” encompasses all polymorphic forms or derivatives thereof.

The dosage forms of the present invention provide similar or comparable in-vitro release of the methylphenidate hydrochloride and are easy to swallow and more patient compliant because of its reduced size and reduced weight in comparison to the reference dosage form.

The term “size” as used herein, means tablet thickness x tablet diameter (a value obtained by multiplication of tablet thickness and tablet diameter).

The term “reduced size” as used herein, means lesser size of the tablets prepared in accordance with the invention in comparison to the reference dosage form. The invention provides reduced size tablet where size is reduced at least by 25%, preferably at least by about 50%, when all approved strengths of reference dosage form are compared with respective strengths of the product disclosed in this invention.

The term “reduced weight” as used herein, means lesser weight of the tablets prepared in accordance with the invention in comparison to the reference dosage form. The invention provides reduced weight tablet where weight of the tablet is reduced at least by about 25%, for example, at least by about 55%.

The term “methylphenidate hydrochloride load” or “MPH load” as used herein, means weight percentage of methylphenidate hydrochloride present in the dosage form. The invention provides high methylphenidate hydrochloride load tablet wherein the load is increased at least by 50%, for example, at least by about 115% in comparison to the reference dosage form.

The term “immediate release” as used herein, means complete release of drug within a time period of about 1 hour or less and, preferably, about 30 minutes or less.

The term “semipermeable membrane” as used herein, means a membrane that allows passage of certain molecules or ions by diffusion while retaining some other molecules, for example, allowing passage of certain fluids such as water or other fluids while retaining drug molecules or osmotic agent molecules.

Inventors have surprisingly found that the extended release osmotic tablet dosage form of methylphenidate hydrochloride can now be prepared having high drug load, reduced size and reduced weight along with maintaining the similar or comparable dissolution profile and bioequivalence parameters in comparison to those of the reference dosage form.

The dosage form of the invention comprises methylphenidate hydrochloride in an amount of about 10 mg to about 80 mg.

In one embodiment, the dosage form of the invention may comprise methylphenidate hydrochloride in an amount of more than about 8%, for example, more than about 9%, more than about 10%, more than about 11%, more than about 12%, more than about 13%, more than about 14%, more than about 15%, more than about 18%, more than about 20% or more than about 22% by weight of the core tablet.

In another embodiment, the dosage form of the invention may comprise methylphenidate hydrochloride in an amount of more than about 8%, for example, more than about 9%, more than about 10%, more than about 11%, more than about 12%, more than about 13%, more than about 14%, more than about 15%, more than about 18%, more than about 20% or more than about 22% by weight of the dosage form.

In yet another embodiment, the dosage form of the invention is in the form of osmotic tablet which comprises multilayer core tablet, for example, a bilayer or a trilayer tablet, a semipermeable membrane surrounding the core tablet having at least one orifice in it, which is further coated with an immediate release drug coat comprising methylphenidate hydrochloride.

In another embodiment of the invention, at least a part of total methylphenidate hydrochloride content is present in the core tablet and remaining part is present in the immediate release drug coat which is coated over the semipermeable membrane. In one embodiment of the invention, the weight ratio of methylphenidate hydrochloride present in the core tablet to the immediate release drug coat is between about 2:1 to about 5:1, for example, about 3.5:1.

In yet another embodiment, amount of methylphenidate hydrochloride present in the dosage form is between about 10 mg to about 80 mg, for example,18 mg, 27 mg, 36 mg, 45 mg, 54 mg, 63 mg and 72 mg.

In yet another embodiment of the invention, the total amount of methylphenidate hydrochloride present in the dosage form is 18 mg, 27 mg, 36 mg, 45 mg, 54 mg, 63 mg or 72 mg, from which respectively 14 mg, 21 mg, 28 mg, 35 mg, 42 mg, 49 mg or 56 mg of methylphenidate hydrochloride is present in the core tablet while remaining amount (respectively 4 mg, 6 mg, 8 mg, 10 mg, 12 mg, 14 mg or 16 mg) of methylphenidate hydrochloride is present in the immediate release drug coat. The core tablet of the present invention may be dose proportional or substantially dose proportional.

In yet another embodiment of the invention, the total amount of methylphenidate hydrochloride present in the dosage form is divided in a way that from about 60% to about 90%, for example about 70%, about 75% or about 80%, of the total amount of methylphenidate hydrochloride is present in the core tablet while remaining amount from about 10% to about 40%, for example about 20%, about 25% or about 30%, of the total amount of methylphenidate hydrochloride is present in the immediate release drug coat.

In another embodiment, the invention provides the extended release osmotic tablet dosage form comprising same amount of the methylphenidate hydrochloride in comparison to the corresponding reference dosage form. The extended release osmotic tablet dosage form according to the present invention comprises 18 mg, 27 mg, 36 mg or 54 mg of the methylphenidate hydrochloride and having reduced size and reduced weight in comparison to the corresponding reference dosage form.

In yet another embodiment, the invention provides the extended release osmotic tablet comprising 18 mg methylphenidate hydrochloride, wherein the diameter of the tablet is between about 4.9 mm-5.1 mm and the thickness of the tablet is less than about 10 mm, for example, less than 8 mm, less than 7 mm or between about 5.8 mm-6.5 mm. Accordingly, the tablet size for the extended release osmotic tablet comprising 18 mg methylphenidate hydrochloride is less than 60 mm², for example less than 55 mm², less than 45 mm², less than 35 mm², or less than 30 mm² or less than 25 mm². Accordingly, the tablet weight for the extended release osmotic tablet comprising 18 mg methylphenidate hydrochloride is less than 290 mg, for example less than 260 mg, less than 230 mg, less than 200 mg, less than 170 mg, less than 140 mg or less than 125 mg.

In yet another embodiment, the invention provides the extended release osmotic tablet comprising 27 mg methylphenidate hydrochloride, wherein the diameter of the tablet is between about 5.1 mm-5.5 mm and the thickness of the tablet is less than about 11 mm, for example, less than 9 mm, less than 8 mm or between about 7.7 mm-8.0 mm. Accordingly, the tablet size for the extended release osmotic tablet comprising 27 mg methylphenidate hydrochloride is less than 62 mm², for example less than 55 mm², less than 50 mm², less than 45 mm², or less than 41 mm². Accordingly, the tablet weight for the extended release osmotic tablet comprising 27 mg methylphenidate hydrochloride is less than 275 mg, for example less than 250 mg, less than 230 mg, less than 210 mg or less than 190 mg.

In yet another embodiment, the invention provides the extended release osmotic tablet comprising 36 mg methylphenidate hydrochloride, wherein the diameter of the tablet is between about 5.8 mm-6.2 mm and the thickness of the tablet is less than about 13 mm, for example, less than 11 mm, less than 9 mm or between about 8.1 mm-8.6 mm.

Accordingly, the tablet size for the extended release osmotic tablet comprising 36 mg methylphenidate hydrochloride is less than 95 mm², for example less than 85 mm², less than 75 mm², less than 65 mm², less than 55 mm² or less than 51 mm². Accordingly, the tablet weight for the extended release osmotic tablet comprising 36 mg methylphenidate hydrochloride is less than 515 mg, for example less than 490 mg, less than 440 mg, less than 400 mg, less than 350 mg, less than 300 mg, less than 270 mg or less than 250 mg.

In yet another embodiment, the invention provides the extended release osmotic tablet comprising 54 mg methylphenidate hydrochloride, wherein the diameter of the tablet is between about 6.4 mm-6.8 mm and the thickness of the tablet is less than about 13.5 mm, for example, less than 12 mm, less than 10.5 mm or between about 9.7 mm-10.1 mm. Accordingly, the tablet size for the extended release osmotic tablet comprising 54 mg methylphenidate hydrochloride is less than 96 mm², for example less than 85 mm², less than 75 mm², less than 65 mm² or less than 62 mm². Accordingly, the tablet weight for the extended release osmotic tablet comprising 54 mg methylphenidate hydrochloride is less than 525 mg, for example less than 490 mg, less than 440 mg, less than 400 mg, less than 380 mg or less than 360 mg.

In yet another embodiment, the invention provides the extended release osmotic tablet comprising 72 mg methylphenidate hydrochloride, wherein the diameter of the tablet is between from about 7.4 mm to about 7.0 mm and the thickness of the tablet is less than about 14.5 mm, for example, less than about 12.5 mm of between about 9.7 mm-10.1 mm. Accordingly, the tablet size for the extended release osmotic tablet comprising 72 mg methylphenidate hydrochloride is less than 125 mm², for example less than 105 mm², less than 95 mm², less than 85 mm² or about 81 mm². Accordingly, the tablet weight for the extended release osmotic tablet comprising 72 mg methylphenidate hydrochloride is less than 600 mg, for example less than 550 mg, less than 530 mg, less than 500 mg or less than 470 mg.

In yet another embodiment, the invention provides the extended release osmotic tablet dosage form comprising 45 mg methylphenidate hydrochloride to provide additional prescribing options and flexibility to the physicians.

In another embodiment, the invention provides an easy to swallow extended release osmotic tablet dosage form comprising methylphenidate hydrochloride in an amount higher than present in the commercially available tablets under the tradename Concerta®, preferably higher than 54 mg methylphenidate hydrochloride is present in the dosage form.

In yet another embodiment, the invention provides an easy to swallow extended release osmotic tablet dosage form comprising 63 mg or 72 mg methylphenidate hydrochloride to provide additional prescribing options and flexibility to the physicians.

In yet another embodiment, the invention provides the extended release osmotic dosage form comprising 72 mg methylphenidate hydrochloride, wherein the diameter of the tablet is between about 6.7 mm-7.5 mm and the thickness of the tablet is less than about 14.5 mm, for example, less than 13 mm, less than 10.5 mm or between about 9.5 mm-10.5 mm.

In yet another embodiment, the dosage form comprises a multilayer core tablet, for example, bilayer tablet or trilayer tablet which comprises at least one drug layer and at least one push layer. In another embodiment, weight ratio of the drug layer to the push layer is between about 0.80:1 to about 1:0.80, more preferably weight ratio is 1:1.

In another embodiment of the invention, the drug layer comprises polyethylene oxide in an amount to provide the weight ratio of methylphenidate hydrochloride to polyethylene oxide present in the drug layer about 1:4 or more, for example, weight ratio is about 1:1.8.

In another embodiment of the invention, the push layer comprises polyethylene oxide in an amount to provide the weight ratio of methylphenidate hydrochloride present in the drug layer to polyethylene oxide present in the push layer is about 1:4.5 or more, for example, weight ratio is about 1:2.1.

In another embodiment, the invention provides the extended release osmotic dosage form which is substantially resistant to dose dumping when administered in the presence of ethanol. The term “substantially resistant to dose dumping when administered in the presence of ethanol” as used herein means that the dosage form retains its extended release properties even when exposed to high concentrations of ethanol. For example, the drug release rate of the dosage form, in an ethanol concentration of 5% as well as at highly concentrated ethanol solution containing 40% ethanol, remains substantially similar to the release rate of the dosage form in an ethanol-free environment. Han Lennernas, “Ethanol-Drug Absorption Interaction: Potential for a Significant Effect on the Plasma Pharmacokinetics of Ethanol Vulnerable Formulations,” Mol. Pharmaceutics, 2009, 6 (5), pp 1429-1440, incorporated by reference in its entirety, recommends a two hour time frame for screening the in vitro dissolution profile of a controlled-release product in ethanol concentrations of up to 40%. Therefore, one definition for resistance to alcohol induced dose dumping is a dosage form that releases less than 60% of the active agent after 30 minutes in an ethanol concentration of at least 40%, less than 70% of the active agent after 60 minutes in an ethanol concentration of at least 40%, and less than 80% of the active agent after 120 minutes in an ethanol concentration of at least 40%.

In yet another embodiment, the invention provides the extended release osmotic dosage form which is substantially resistant to dose dumping when administered in the presence of ethanol, provides similar drug release profile to that of the reference dosage form containing respective amount of drug.

The dosage form comprises pharmaceutically acceptable excipients including one or more fillers, binders, disintegrants, lubricants, glidants, release rate controlling polymers, osmogents (osmotic agents), anti-oxidants, pH modifiers, coating polymers, pore formers, colorants, wetting agents, plasticizers etc.

Suitable fillers may include one or more of microcrystalline cellulose, starch, dibasic calcium phosphate, tribasic calcium phosphate, calcium carbonate, dextrose, kaolin, magnesium carbonate, magnesium oxide; sugars such as lactose or sucrose; The filler may be present in an amount of 5% to 90% by weight of the dosage form.

Suitable binders may include one or more of hydroxyethyl cellulose, hydroxypropyl cellulose, hydroxypropyl methylcellulose, carbomer, dextrin, ethyl cellulose, methylcellulose, shellac, zein, gelatin, polymethacrylate (e.g. eudragit), polyvinylpyrrolidone, pregelatinized starch, sodium alginate, gums, synthetic resins, silicic acid, hydrophilic polymer and the like. The binder may be present in an amount of about 1% to about 20% by weight of the dosage form.

Suitable disintegrants may include one or more of croscarmellose sodium, sodium starch glycolate, low substituted hydroxylpropyl cellulose (L-hydroxylpropyl cellulose), pregelatinized starch, sodium carboxymethyl cellulose, cross-linked polyvinylpyrrolidone and the like. The disintegrants are present in an amount of 0.5% to 20% by weight of the dosage form.

Suitable lubricants and glidants may include one or more of talc, metallic stearates such as magnesium stearate, calcium stearate, zinc stearate; colloidal silicon dioxide, finely divided silicon dioxide, stearic acid, hydrogenated vegetable oil, glyceryl palmitostearate, glyceryl monostearate, glyceryl behenate, polyethylene glycols, powdered cellulose, sodium stearyl fumarate, sodium benzoate, mineral oil, magnesium trisilicate, kaolin; and the like. It would be appreciated that a person skilled in the art is cognizant of the fact that lubricant, glidant or anti-tacking agent may be used interchangeably. The lubricant, glidant or anti-tacking agent may be present in an amount ranging from 0.1% to 10% by weight of the dosage form.

The term “release rate controlling polymers” encompasses one or more polymers which are able to control the release of methylphenidate hydrochloride from the dosage form. Suitable release rate controlling polymers may include one or more of polyethylene oxide, cellulose derivatives, in particular HPMC, HPC, carboxymethylcellulose, poly(oxyethylene) alkyl ether, high viscosity grade polyethylene glycol, co-block polymers of ethylene oxide, and propylene oxide (poloxamer, pluronic), derivatives of polymethacrylates, polyvinyl alcohol, polyvinyl alcohol derivatives, polyethylene glycol, and polyethylene glycol derivatives. In one specific embodiment, preferable release rate controlling polymer to be used in accordance with the invention is polyethylene oxide. In one preferred embodiment of the invention, the dosage form may include one or more release rate controlling polymers, preferably two different release rate controlling polymers. In some embodiments of the invention, hydroxypropylmethyl cellulose may be used alone or in addition to polyethylene oxide.

In some embodiments of the invention, one or more release rate controlling polymers are present in the drug layer or in the push layer or in both the layers.

In one preferred embodiment, the invention provides a dosage form comprising suitable amount of polyethylene oxide and hydroxypropylmethyl cellulose both present in the drug layer as well as in the push layer.

In another preferred embodiment of the invention, the release rate controlling polymer is polyethylene oxide, present in the dosage form in an amount to provide weight ratio of methylphenidate hydrochloride to polyethylene oxide present in the dosage form is about 1:6.6 or more, preferably the weight ratio is about 1:3.

In another preferred embodiment of the invention, the release rate controlling polymer is polyethylene oxide, present in the core tablet in the amount to provide weight ratio of methylphenidate hydrochloride to polyethylene oxide present in the core tablet is about 1:8.5 or more, preferably weight ratio is about 1:4.

In another embodiment, the dosage form comprising high methylphenidate hydrochloride load, reduced size and reduced weight may be made by means of increasing the weight ratio for methylphenidate hydrochloride to release rate controlling polymer.

Suitable osmogents may include one or more of inorganic salts such as NaCl, KCl and the like. Preferably, the invention provides a dosage form which does not contain polyol sugar osmogent like mannitol, sorbitol and the like. The osmogent may present in an amount from 10% to 20% by weight of the core tablet, for example, 15% or 14%.

Suitable anti-oxidants may include one or more of butylated hydroxy toluene, butylated hydroxy anisole, propyl gallate, vitamin E and the like.

Suitable pH modifiers may include one or more of organic acids such as oxalic acid, malonic acid, succinic acid, glutaric acid, adipic acid, pimelic acid, suberic acid, azelaic acid, sebacic acid, brassylic acid, thapsic acid, formic acid, acetic acid, propionic acid, butyric acid, valeric acid, caproic acid, malic acid, benzoic acid, carbonic acid and the like.

Suitable coating polymers may include one or more of alkyl celluloses, hydroxyalkyl celluloses, cellulose ethers, cellulose esters, nitro celluloses, polymers of acrylic and methacrylic acids and esters thereof, polyamides, polycarbonates, polyalkylenes, polyalkylene glycols, polyalkylene oxides, polyalkylene terephthalates, polyvinyl alcohols, polyvinyl ethers, polyvinyl esters, cellulose acetate, cellulose acetate butyrate, agar acetate, amylose triacetate, betaglucan acetate, poly (vinyl methyl) ether copolymers, poly (orthoesters), polyacetals, poly (glycolic acid), poly (lactic acid) and derivatives and/or co-polymers thereof and the like. Preferably coating polymers may include one or more of hydroxypropylmethyl cellulose (HPMC), hydroxypropyl cellulose, hydroxyethyl cellulose, ethyl cellulose and the like.

Suitable polymers used for the preparation of semipermeable membrane may include one or more of cellulose acetate, cellulose acetate butyrate, agar acetate, amylose triacetate, betaglucan acetate, poly (vinyl methyl) ether copolymers, poly (orthoesters), polyacetals, poly (glycolic acid) and poly (lactic acid) derivatives and the like.

In another embodiment of the invention, the semipermeable membrane is having one or more orifice created using suitable drilling technique to provide release of the drug from the core tablet.

Suitable pore formers may include one or more of surfactants like poloxamers, sodium lauryl sulfate, sodium dodecyl sulfate, sodium dodecyl benzene sulfonate, sorbitan monoester etc., sugars like glucose, lactose, maltose, sucrose etc., sugar alcohols like erythritol, mannitol, sorbitol etc., water soluble ingredients like hydroxypropylmethyl cellulose, polyethylene glycol, cellulose derivatives, polyvinylpyrrolidone, hydrogenated castor oil, lecithin etc. The weight ratio of the polymer used for preparation of the semipermeable membrane to the pore former present in the semipermeable membrane is between 85:15 and 95:05, for example, 90:10.

In another embodiment, the invention provides a process for preparation of the extended release osmotic tablet dosage forms.

In another embodiment, the invention provides a process for preparation of granules for the drug layer and the push layer. The granules may be prepared using conventional wet granulation, dry granulation or melt granulation method, preferably wet granulation using the fluid bed granulation technique.

In yet another embodiment of the invention, direct compression may be used for the preparation of core tablets comprising the drug layer and the push layer.

In another embodiment, the invention provides a process for longitudinal compression of the drug layer and push layer to provide longitudinally compressed tablet (LCT). Preferably, the bilayer core tablets of the present invention are configured such that each of the drug layer and push layer is substantially round in cross-dimension. The two layers are compressed together longitudinally such that the resulting bilayer core tablet has the same circumferential width as that of the drug layer and the push layer. The tablet shape can be described as “capsule shaped” wherein the bilayer core tablet has a circumferential width that is less than its thickness and has a rounded “narrow” top end and a rounded “narrow” bottom end.

In another embodiment, the invention provides a process for applying a semipermeable membrane surrounding the core tablet using suitable excipients and conventional coating techniques.

In another embodiment, the invention provides a method for creating an orifice having diameter of around 0.6±0.2 mm and depth of 0.5±0.2 mm for the drug release by drilling through the semipermeable membrane at the narrow end of the compartment proximate to the drug layer. Preferably, suitable conventional drilling techniques (e.g. laser drilling) may be used for creating an orifice through the semipermeable membrane.

In yet another embodiment, the invention provides a process for applying an immediate release drug coat comprising methylphenidate hydrochloride over the semipermeable membrane. The immediate release drug coat may be prepared by spraying the solution comprising methylphenidate hydrochloride and hydrophilic film forming polymer dissolved in suitable solvent on to the semipermeable membrane.

In another embodiment, the invention provides a method for treatment of Attention Deficit Hyperactivity Disorder (ADHD) comprising administering to the patient in need thereof, the extended release osmotic tablet dosage forms comprising methylphenidate hydrochloride as per the invention. ADHD is more prevalent in children and adolescent, who may face difficulties in swallowing the tablet. For such patient population, reduced size tablets of the present invention, provides ease of administration and hence patient compliance can be achieved.

In another embodiment of the invention, the extended release osmotic tablet dosage form comprising methylphenidate hydrochloride provides similar or comparable dissolution profile in comparison to the reference dosage form. For example, the dosage form of the present invention provides at least about 15% of the MPH release within first one hour, at least about 50% of the MPH release within first six hours and at least about 80% of the MPH release within first eight hours of the dissolution testing. The media used for the dissolution testing was 50 ml, acidified water (US FDA Office of Generic Drug (OGD) recommended dissolution media) adjusted with phosphoric acid to a pH of 3. The apparatus used for the dissolution testing was USP type VII at 30 dpm (dips per minute) and method used was USP Test 2 as per the USP monograph for Methylphenidate Hydrochloride Extended Release Tablets.

In yet another embodiment of the invention, the extended release osmotic tablet dosage form comprising methylphenidate hydrochloride provides similar or comparable bioequivalence parameters like Cₘₐₓ and AUC_{0-∞} in comparison to that of the reference dosage form. The extended release osmotic tablet dosage form comprising methylphenidate hydrochloride provides bioequivalence parameters like AUC_{0-∞} and Cₘₐₓ in the range of 80% to 125%, in comparison to that of the reference dosage form.

The extended release osmotic tablet dosage of the invention provides an in-vivo plasma profile for methylphenidate hydrochloride when administered in a fasted state comprising:
a mean of Cₘₐₓ from about 10 ng/mL to about 25 ng/mL and
a mean of AUC_{0-∞} from about 150 ng*hr/mL to about 300 ng*hr/mL.

The extended release osmotic tablet dosage form of the invention provides an in-vivo plasma profile for methylphenidate hydrochloride when administered in a fed state comprising:
a mean of Cₘₐₓ from about 15 ng/mL to about 30 ng/mL and
a mean of AUC_{0-∞} from about 225 ng*hr/mL to about 325 ng*hr/mL.

The invention is illustrated by the below examples which are provided to be exemplary of the invention and do not limit the scope of the invention.

### EXAMPLE 1

### Extended Release Osmotic Tablet

TABLE 1A

| | |
|---|---|
| Ingredient | Amount (mg) |

Drug layer

| | |
|---|---|
| Methylphenidate hydrochloride | 42.00 |
| Polyethylene oxide | 74.42 |
| Succinic acid | 6.30 |
| Povidone | 5.60 |
| Butylated hydroxy toluene | 0.08 |
| Iron oxide red | 0.60 |
| Hypromellose | 4.50 |
| Stearic acid | 1.50 |
| Isopropyl alcohol and water | q.s. |

Push layer

| | |
|---|---|
| Polyethylene oxide | 89.04 |
| Sodium chloride | 39.40 |
| Hypromellose | 7.50 |
| Povidone | 7.50 |
| Butylated hydroxy toluene | 0.06 |
| Stearic acid | 1.50 |
| Isopropyl alcohol and water | q.s. |
| Weight of the core tablet | 280.0 |

Semipermeable membrane

| | |
|---|---|
| Cellulose acetate | 27.37 |
| Poloxamer | 4.83 |
| Acetone | q.s. |
| Water | q.s. |

Immediate release drug coat

| | |
|---|---|
| Methylphenidate hydrochloride | 12.00 |
| Succinic acid | 1.800 |
| Opadry | 16.20 |
| Purified water | q.s. |

Aesthetic film coat

| | |
|---|---|
| Opadry | 17.11 |
| Total weight of the dosage form | 359.31 |

Process:

Granules for preparation of the drug layer were prepared by granulating the powder mixture of methylphenidate hydrochloride, polyethylene oxide, iron oxide red and succinic acid using binder solution containing povidone & butylated hydroxy toluene dissolved in mixture of isopropyl alcohol and water. Prepared granules were dried and dried granules were sifted to get appropriate dimension granules which were blended with hypromellose and further lubricated with stearic acid.

Granules for preparation of the push layer were prepared by granulating the powder mixture of polyethylene oxide, sodium chloride and hypromellose using the binder solution containing povidone & butylated hydroxy toluene dissolved in mixture of isopropyl alcohol and water. Prepared granules were dried and the dried granules were sifted to get appropriate dimensions which were further lubricated with stearic acid.

Granules prepared in the above steps for the drug layer and the push layer were compressed using the bilayer compression machine with appropriate tooling to provide bilayer longitudinally compressed core tablets.

A semipermeable membrane was formed surrounding the core tablets. Cellulose acetate & poloxamer 188 were dissolved in acetone:water mixture to prepare a solution which was then sprayed over the bilayer core tablets.

A suitable orifice for the drug release was drilled through the semipermeable membrane at the narrow end of the compartment proximate to the drug layer to thereby form the extended release osmotic bilayer tablets.

An immediate release drug coat comprising methylphenidate hydrochloride was additionally coated over the semipermeable membrane. Methylphenidate hydrochloride, succinic acid and opadry were dissolved in purified water and thus prepared solution was sprayed over the semipermeable membrane to provide an immediate release drug coat.

An aesthetic film coat was applied over the immediate release drug coat by spraying the dispersion of opadry in purified water over the immediate release drug coat.

TABLE 1B
Dissolution data were determined periodically every hour
for eight to ten hours using in vitro dissolution testing
apparatus and method. Media: Acidified water adjusted with
phosphoric acid to a pH of 3 (OGD Media), USP type VII,
50 ml, 30 dpm (USP Test 2)

| | Cumulative percentage drug | |
|---|---|---|
| | release from the reference | Cumulative percentage drug |
| | dosage form | release from osmotic tablet |
| Time in hour | (54 mg Concerta ®) | prepared as per Example 1 |
| 1 | 24 | 22 |
| 2 | 29 | 29 |
| 3 | 37 | 39 |
| 4 | 48 | 49 |
| 5 | 60 | 61 |
| 6 | 72 | 72 |
| 7 | 84 | 83 |
| 8 | 95 | 93 |
| 10 | 100 | 96 |

One set of human volunteers were orally administered with the reference tablet which is Concerta®, 54 mg methylphenidate hydrochloride, extended release tablets in fasted condition, and another set of human volunteers were orally administered with the test tablet prepared according to Example 1 in fasted condition. The pharmacokinetic parameters such as Cₘₐₓ, & AUC_{0-∞} were measured and mentioned in Table 1C below. From the data mentioned in Table 1C, it is evident that, test tablet is bioequivalent to the reference tablet in fasted condition. From the data mentioned in Table 1C, it is also evident that, the test tablet meets the current bioequivalence recommendations for generic drug development provided by the USFDA in the fasted condition.

TABLE 1C

| | REFERENCE | TEST |
|---|---|---|
| PARAMETER | GEOMETRIC MEAN | GEOMETRIC MEAN |
| Cₘₐₓ | 18.853 | 17.406 |
| (ng/mL) | | |
| AUC_{0-∞} | 221.954 | 202.738 |
| (ng * hr/mL) | | |

One set of human volunteers were orally administered with the reference tablet which is Concerta®, 54 mg methylphenidate hydrochloride, extended release tablets in fed condition, and another set of human volunteers were orally administered with the test tablet prepared according to Example 1 in fed condition. The pharmacokinetic parameters such as Cₘₐₓ, & AUC_{0-∞} were measured and mentioned in Table 1D below. From the data mentioned in Table 1D, it is evident that, test tablet is bioequivalent to the reference tablet in the fed condition. From the data mentioned in Table 1D, it is also evident that, test tablet meets the current bioequivalence recommendations for generic drug development provided by USFDA in the fed condition.

TABLE 1D

| | REFERENCE | TEST |
|---|---|---|
| PARAMETER | GEOMETRIC MEAN | GEOMETRIC MEAN |
| Cₘₐₓ | 22.016 | 20.596 |
| (ng/mL) | | |
| AUC_{0-∞} | 284.152 | 262.618 |
| (ng * hr/mL) | | |

### EXAMPLE 2

### Extended Release Osmotic Tablet

TABLE 2A

| | |
|---|---|
| Ingredient | Amount (mg) |

Drug layer

| | |
|---|---|
| Methylphenidate hydrochloride | 14 |
| Polyethylene oxide | 24.8 |
| Succinic acid | 2.10 |
| Povidone | 1.86 |
| Butylated hydroxy toluene | 0.02 |
| Iron oxide red | 0.20 |
| Hypromellose | 1.50 |
| Stearic acid | 0.50 |
| Isopropyl alcohol and water | q.s. |

Push layer

| | |
|---|---|
| Polyethylene oxide | 29.68 |
| Sodium chloride | 13.13 |
| Hypromellose | 2.50 |
| Povidone | 2.50 |
| Butylated hydroxy toluene | 0.02 |
| Stearic acid | 0.50 |
| Isopropyl alcohol and water | q.s. |
| Weight of the core tablet | 93.33 |

Semipermeable membrane

| | |
|---|---|
| Cellulose acetate | 12.600 |
| Poloxamer | 1.400 |

Immediate release drug coat

| | |
|---|---|
| Methylphenidate hydrochloride | 4 |
| Succinic acid | 0.60 |
| Opadry | 5.40 |
| Purified water | q.s. |

Aesthetic film coat

| | |
|---|---|
| Opadry | 5.86 |
| Total weight of the dosage form | 123.20 |

Process:

Extended release osmotic tablets were made in accordance with the manufacturing processes of Example 1 using the ingredients and their amounts as per the Table 2A.

Extended release osmotic tablets prepared according to the Example 2 were measured for their dimensions and weights, and their comparison with the corresponding commercially available tablets is given in Table 2B below.

TABLE 2B

| | Reference | | |
|---|---|---|---|
| | dosage form | | |
| | (18 mg | Tablets of | |
| Parameters | Concerta ®) | Example 2 | Percentage change |
| Tablet Diameter | 5.30 | 5.00 | 5.67% reduction in |
| (mm) | | | Diameter |
| Tablet Thickness | 11.50 | 6.10 | 47.74% reduction in |
| (mm) | | | Thickness |
| Tablet Size | 60.95 | 30.5 | 50% size reduction |
| (mm²) | | | |
| Tablet Weight | 267.00 | 123.20 | 53.86% reduction in |
| (mg) | | | weight |
| Tablet MPH load | 6.74 | 14.61 | 117% increase in MPH |
| | | | load |

As per the results in Table 2B, it is revealed that the thickness of the tablets made in accordance with the present invention is reduced approximately by 47% in comparison to that of the reference dosage form. Thus one preferred aspect of the invention provides reduced size and hence easy to swallow dosage forms containing high drug load.

As per the results in Table 2B, it is revealed that the weight of the tablets made in accordance with the present invention is reduced approximately by 54% in comparison to that of the reference dosage form. Thus, in one preferred aspect of the invention provides reduced weight tablet dosage forms containing high drug load.

Study for evaluation of dose dumping resistance in presence of alcohol: Tablets prepared according to example 1 (test) and the tablets of reference dosage form [Concerta® Extended Release Tablets, 54 mg] were tested for its dose dumping resistance in presence of alcohol (ethanol) using USP type 2 apparatus, at 50 RPM speed and using 900 ml of 0.1N HCl aqueous solution as media. The results of the said trials are mentioned in Table 3 as below.

TABLE 3

| | 0.1N HCl + | | 0.1N HCl + | | 0.1N HCl + | |
|---|---|---|---|---|---|---|
| Time | ethanol free | | 5% ethanol | | 40% ethanol | |
| (min) | Reference | Test | Reference | Test | Reference | Test |
| 15 | 21 | 17 | 21 | 18 | 17 | 16 |
| 30 | 23 | 19 | 23 | 19 | 21 | 19 |
| 45 | 23 | 20 | 23 | 21 | 24 | 21 |
| 60 | 24 | 21 | 24 | 22 | 27 | 23 |
| 75 | 25 | 22 | 25 | 24 | 30 | 27 |
| 90 | 26 | 24 | 26 | 26 | 34 | 30 |
| 105 | 27 | 26 | 28 | 29 | 37 | 36 |
| 120 | 28 | 28 | 29 | 31 | 42 | 41 |
| F2 | NA | 76 | NA | 83 | NA | 77 |

From the data mentioned in Table 3, it is evident that, the test tablets are dose dumping resistant in presence of alcohol and comparable to the reference dosage form.

While the present invention has been described in terms of its specific embodiments, certain modifications and equivalents will be apparent to those skilled in the art and are intended to be included within the scope of the present invention.

1. An extended release osmotic tablet dosage form comprising:
i. a bilayer core tablet comprising methylphenidate hydrochloride in an amount of more than 8% by weight of the core tablet,
ii. a semipermeable membrane surrounding the core tablet, and
iii. an immediate release drug coat comprising methylphenidate hydrochloride surrounding the semipermeable membrane.

2. The extended release osmotic tablet dosage form according to claim 1, wherein the semipermeable membrane has at least one orifice for release of methylphenidate hydrochloride from the core tablet.

3. The extended release osmotic tablet dosage form according to claim 1, wherein the amount of methylphenidate hydrochloride present in the core tablet is more than about 10% by weight of the core tablet.

4. The extended release osmotic tablet dosage form according to claim 1, wherein the amount of methylphenidate hydrochloride present in the core tablet is more than about 12% by weight of the core tablet.

5. The extended release osmotic tablet dosage form according to claim 1, wherein the amount of methylphenidate hydrochloride present in the core tablet is about 15% by weight of the core tablet.

6. The extended release osmotic tablet dosage form according to claim 1, wherein ratio of methylphenidate hydrochloride present in the core tablet to methylphenidate hydrochloride present in the immediate release drug coat is between about 2:1 and about 5:1.

7. The extended release osmotic tablet dosage form according to claim 1, wherein the dosage form comprises from about 10 mg to about 80 mg of methylphenidate hydrochloride.

8. (canceled)

9. The extended release osmotic tablet dosage form according to claim 1, wherein the core tablet comprises a drug layer and a push layer.

10. The extended release osmotic tablet dosage form according to claim 9, wherein weight ratio of the drug layer to the push layer is between about 0.8:1 and about 1:0.8.

11. The extended release osmotic tablet dosage form according to claim 10, wherein the weight ratio is about 1:1.

12. The extended release osmotic tablet dosage form according to claim 1, wherein the core tablet dosage form comprises at least one release rate controlling polymer.

13. The extended release osmotic tablet dosage form according to claim 12, wherein the release rate controlling polymer is polyethylene oxide.

14. The extended release osmotic tablet dosage form according to claim 13, wherein weight ratio of methylphenidate hydrochloride present in the dosage form to polyethylene oxide present in the dosage form is about 1:6.6 or more.

15. The extended release osmotic tablet dosage form according to claim 14, wherein the weight ratio is about 1:3.

16. The extended release osmotic tablet dosage form according to claim 13, wherein weight ratio of methylphenidate hydrochloride present in the core tablet to polyethylene oxide present in the core tablet is about 1:8.5 or more.

17. The extended release osmotic tablet dosage form according to claim 16, wherein the weight ratio is about 1:4.

18. The extended release osmotic tablet dosage form according to claim 1, wherein the dosage form provides an in-vivo plasma profile for methylphenidate hydrochloride when administered in a fasted state comprising:
a mean of Cₘₐₓ from about 10 ng/mL to about 25 ng/mL, and
a mean of AUC_{0-∞} from about 150 ng*hr/mL to about 300 ng*hr/mL.

19. The extended release osmotic tablet dosage form according to claim 1, wherein the dosage form provides an in-vivo plasma profile for methylphenidate hydrochloride when administered in a fed state comprising:
a mean of Cₘₐₓ from about 15 ng/mL to about 30 ng/mL, and
a mean of AUC_{0-∞} from about 225 ng*hr/mL to about 325 ng*hr/mL.

20. An extended release osmotic tablet dosage form comprising about 18 mg methylphenidate hydrochloride, wherein the tablet has size less than 60 mm² and weight less than 260 mg.

21. An extended release osmotic tablet dosage form comprising about 27 mg methylphenidate hydrochloride, wherein the tablet has size less than 62 mm² and weight less than 275 mg.

22. An extended release osmotic tablet dosage form comprising about 36 mg methylphenidate hydrochloride, wherein the tablet has size less than 95 mm² and weight less than 515 mg.

23. An extended release osmotic tablet dosage form comprising about 54 mg methylphenidate hydrochloride, wherein the tablet has size less than 96 mm² and weight less than 525 mg.

24. An extended release osmotic tablet dosage form comprising about 72 mg methylphenidate hydrochloride, wherein the tablet has size less than 125 mm² and weight less than 600 mg.
